(19) United States
(12) Patent Application Publication
(10) Pub. No.: US 2025/0129229 A1
(43) Pub. Date: Apr. 24, 2025

(54) Foam Comprising Thermoplastic Polymers and Expanded Microspheres Suitable for Medical Tapes and Bandages

(71) Applicant: 3M INNOVATIVE PROPERTIES COMPANY, St. Paul, MN (US)

(72) Inventors: Diane L. Emslander, City of Grant, MN (US); Jeffrey O. Emslander, City of Grant, MN (US); Lacey M. Strand, St. Paul, MN (US); Jabob D. Young, St. Paul, MN (US); Aniruddha A. Upadhye, St. Paul, MN (US); Jessica L. Petersen, Stillwater, MN (US)

(21) Appl. No.: 18/888,368

(22) Filed: Sep. 18, 2024

Related U.S. Application Data
(60) Provisional application No. 63/591,565, filed on Oct. 19, 2023.

(51) Int. Cl. C08J 9/32; C08K 9/10; C09J 7/38

(52) U.S. Cl. CPC C08J 9/32; C08K 9/10; C09J 7/385; C08J 2203/22; C08J 2207/02; C08J 2300/26; C08J 2375/08; C09J 2301/302; C09J 2301/412

(57) ABSTRACT

An article is described comprising a foam substrate comprising: a thermoplastic composition comprising polyurethane, a copolymer of ethylene and a polar comonomer, or a combination thereof; and expanded microspheres; and wherein the article is a medical tape, bandage or wrap. Also describes are methods of use, methods of making a foam article, foam substrates comprising a pressure sensitive adhesive layer disposed on a outermost major surface of the foam substrate, and foam compositions.

### SUMMARY

In one embodiment, an article is described comprising a foam substrate comprising: a thermoplastic composition comprising polyurethane, a copolymer of ethylene and a polar comonomer, or a combination thereof; and expanded microspheres; and wherein the article is a medical tape, bandage or wrap. The foam preferably has a MVTR of at least 100, 200, 300, 400, 500, or 600 g/m² per 24 hours.

In some embodiments, the foam has a Shore A hardness ranging from 40-50. In some embodiments, the foam is waterproof. In some embodiments, the thermoplastic composition comprises polyurethane and a copolymer of ethylene and a polar comonomer. In some embodiments, the article further comprises a pressure sensitive adhesive skin contact adhesive disposed on a major surface of the foam substrate.

In another embodiment, a method of use is described comprising providing a foam article as described herein and contacting the foam and/or pressure sensitive adhesive with skin.

In another embodiment, a method of making a foam article is described comprising: a) providing a composition comprising a thermoplastic polymer comprising polyurethane, a copolymer of ethylene and a polar comonomer, or a combination thereof; and expandable microspheres having a minimum onset temperature; and b) extruding the composition at a temperature greater than the minimum onset temperature of the expandable microspheres. In some embodiments, the composition further comprises a blowing agent.

In another embodiment, an article is described comprising a foam substrate comprising a thermoplastic polymer comprising polyurethane, a copolymer of ethylene and a polar comonomer, or a combination thereof; and expanded microspheres; and a pressure sensitive adhesive layer disposed on a outermost major surface of the foam substrate.

In another embodiments, an article is described comprising foam, wherein the foam comprises a thermoplastic polymer comprising polyurethane, a copolymer of ethylene and a polar comonomer, or a combination thereof; expanded microspheres; and chemical blowing agent.

### BRIEF DESCRIPTION OF THE DRAWINGS

FIG. 1 is an illustrative cross-section of an embodied article comprising at least two foam layers comprising expanded microspheres;

FIG. 2 is an illustrative cross-section of an embodied article comprising at least two foam layers comprising expanded microspheres and optional internal adhesive layers;

FIG. 3 is an illustrative cross-section of an embodied article comprising a foam layer comprising expanded microsphere and a pressure sensitive adhesive layer disposed on a major surface of the foam layer.

### ARTICLE

The present invention is related to articles comprising one or more foam layers. The foam layer(s) can have a moisture vapor transmission rate that is suitable for medical articles such as medical adhesive tapes and adhesive wound dressings (e.g. bandage). In favored embodiments, the foam is also waterproof.

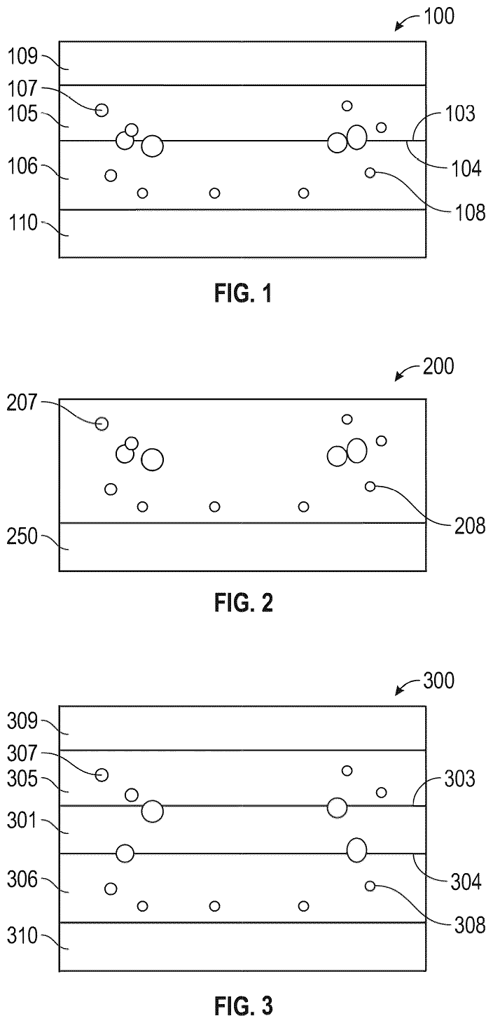

FIG. 1 is a schematic depiction of a cross-section of an illustrative (e.g. medical) article 100 that includes two foam layers, i.e. first foam layer 105 and second foam layer 106. In some embodiments, the polymeric multilayer material may comprise more than two foam layers. For example, the polymeric multilayer material may comprise at least 3, 4, or 5 foam layers.

When the foam layers (e.g. 105 and 106) contain the same composition, the foam layers may form a single homogenous foam layer, as illustrated in FIG. 2. In other words, there may not be a distinguishable interface between two or more foam layers.

With reference to FIG. 3, illustrative (e.g. medical) article 300 may optionally comprise an internal adhesive layer 301 between foam layers, such as described in U.S. Pat. No. 11,097,508. In some embodiments, an adhesive layer may be present between each adjacent foam layers. When present, the optional adhesive layer may be present at a thickness of at least 2, 3, 4, or 5% of the total polymeric multilayer material thickness. In some embodiments, the optional adhesive layer may be present at a thickness of no greater than 20, 15, or 10% of the total polymeric multilayer material thickness). The adhesive layer can have a thickness in a range from 5 micrometers to 250 micrometers (in some embodiments, 10 micrometers to 150 micrometers, 15 micrometers to 100 micrometers, or even 20 micrometers to 40 micrometers). Such internal adhesive layers may also be described as a tie layer. Such internal adhesive layers are not present on the outermost surface of the polymeric multilayer material. In some embodiments, the polymeric multilayer material lacks internal adhesive layers.

In some embodiments, such as during manufacture, the foam layer(s) of the illustrative (e.g. medical) article 100 or 300 may further comprise outer layers, i.e. first outer layer 109, 309 and second outer layer 110, 310. In some embodiments, the outer layer(s) may be present at a thickness of at least 2, 3, 4, 5, 6, 7, 8, 9, or 10% of the total thickness of the foam layer(s). In some embodiments, the outer layer(s) may be present at a thickness of no greater than 40, 35, 30, 25, or 20% of the total thickness of the foam layer(s). The thickness of an individual outer layer (e.g. 109 and/or 110) can be at least 5, 10, 15, 20, or 25 microns. In some embodiments, the thickness of an individual outer layer (109, 309 and/or 110, 310) is no greater than 250, 200, 150, 100, or 50 micrometers. One or both outer layers may be removed after manufacture.

In some embodiments, after removal of one or both outer layers, a pressure sensitive adhesive layer 250 may be disposed on an outermost surface of the foam layer(s), as depicted in FIG. 2. The pressure sensitive adhesive layer 250 may have thickness in the same range as individual outer layer (e.g. 109 and/or 110), as previously described.

One or more of the foam layers comprises expanded microspheres (e.g. 107, 108, 207, 208, 307, 308). In typical embodiments, the one or more foam layers also comprises open and/or closed cells that are derived from blowing agents.

Although the foam can be used for other types of adhesive articles, including tape, the properties of the foam are particularly favorable for medical articles including medical tapes, bandages, and wraps (i.e. self-adhesive bandage wrap to hold gauze and dressings in place). In typical embodiments, such articles are utilized in methods that comprise contacting the foam and/or pressure sensitive adhesive with (e.g. human or animal) skin and underlying tissue.

Medical articles may comprise other optional components such as absorbent pads, backings, and facings layers, such as described in US2019/0231604 incorporated herein by reference.

### Thermoplastic Polyurethane (TPU)

In some embodiments, the one or more foam layers comprise a thermoplastic polyurethane.

Thermoplastic polyurethanes are obtainable by reacting a difunctional isocyanate composition with at least one difunctional polyhydroxy compound and optionally a chain extender. The difunctional isocyanate composition may comprise any aliphatic, cycloaliphatic or aromatic isocyanates. In some embodiments, aliphatic isocyanates can be preferred for obtaining lower Shor A hardness.

The difunctional polyhydroxy compound typically comprises one or more polyether diols, optionally in combination with other diols, such as polyester diols.

Common polyether diols include for example ethylene oxide, propylene oxide, butylene oxide or tetrahydrofuran in the presence, where necessary, of difunctional initiators. Suitable initiator compounds contain two active hydrogen atoms and include water, butanediol, ethylene glycol, propylene glycol, diethylene glycol, triethylene glycol, dipropylene glycol, 1,3-propane diol, neopentyl glycol, 1,4-butanediol, 1,5-pentanediol, 1,6-pentanediol and the like.

Polyester diols which may be used include hydroxyl-terminated reaction products of dihydric alcohols and dicarboxylic acids or their ester-forming derivatives. Common dihydric alcohols include for example ethylene glycol, propylene glycol, diethylene glycol, 1,4-butanediol, neopentyl glycol, 2-methylpropanediol, 3-meth-ylpentane-1,5-diol, 1,6-hexanediol or cyclohexane dimethanol or mixtures. Common dicarboxylic acids or their ester-forming derivatives, include for example succinic, glutaric and adipic acids or their dimethyl esters, sebacic acid, phthalic anhydride, tetrachlorophthalic anhydride or dimethyl terephthalate or mixtures thereof.

Physical properties of suitable thermoplastic polyurethanes are described in the following table:

| | | | Elastollan 1385 A | Estane 58245 is an |
|---|---|---|---|---|
| | Elastollan 1170 A | | polyether and | aromatic polyether |
| Thermoplastic | polyether polyurethane | | polyester polyurethane | polyurethane (high |
| Polyurethane | First PUR - lower Shore | | Second PUR - higher | moisture vapor |
| Property | Hardness | | MVTR | transmission) |
| Density (ISO | 1.08 | | 1.21 | 1.21 |
| 1183/A) | | | | |
| Tensile Stress | 30 | MPa | 30 MPa | 31 MPa |
| (Yield) (DIN | | | | |
| 53504-S2) | | | | |
| Elongation at | 800% | | 750% | 700% |
| Break | | | | |
| Shore Hardness | 71 | | 85 | 80 Shore A |
| (Shore A, 3 sec) | | | | |
| (ISO 7619) | | | | |
| Processing (Melt) | 200-210° | C. | 175-220° C. for | Tm (by DSC) 275° F. |

| | | | |
|---|---|---|---|
| Temp | | extrusion | (135° C.) |
| water vapor | 388 g/m² per 24 hours | 786 g/m² per 24 hours | 650 g/m² per 24 hours |
| permeability | | | MVT Upright Cup |
| (WDD) | | | (ASTM E96 B) |
| (DIN53122-2, | | | |
| 23° C. at 85% | | | |
| relative humidity | | | |
| for a 50 micron | | | |
| film | | | |

The thermoplastic polyurethane typically has a melt processing temperature of at least 125, 150, 175, or 200° C. In some embodiments, the thermoplastic polyurethane typically has a melt processing temperature of no greater than 250, 240, 220, 200, 190, or 180° C. The thermoplastic polyurethane typically has an elongation at break of at least 500, 600, 700, or 800%. In some embodiments, the thermoplastic polyurethane has an elongation at break of no greater than 1000, 900, 800, or 700%. In some embodiments, the thermoplastic polyurethane typically has a tensile stress of at least 20, 25 or 30 MPa. In some embodiments, the thermoplastic polyurethane typically has a tensile stress of no greater than 50 or 40 MPa.

The thermoplastic polyurethane typically contributes to the Shore A hardness of the foam. In some embodiments, the thermoplastic polyurethane typically has a Shore A hardness of no greater than 90, 85, 80, or 75. In some embodiments, the thermoplastic polyurethane has a Shore A hardness of at least 70.

The thermoplastic polyurethane typically contributes to the MVTR properties of the foam. In some embodiments, the (i.e. unfoamed) thermoplastic polyurethane polymer has a water vapor permeability (WDD) of at least 300, 400, 500, 600, 700, or 800 g/m² per 24 hours. In some embodiments, the (i.e. unfoamed) thermoplastic polyurethane polymer has a water vapor permeability (WDD) no greater than 1000, 900, 800, 700, 600, or 500 g/m² per 24 hours.

In some embodiments, a blend of at least two thermoplastic polyurethane polymers is utilized.

In some embodiments, a first thermoplastic polyurethane polymers, such as Elastollan 1170 or a polymer having similar properties to such thermoplastic polyurethane (i.e. Elastollan 1170 type TPU), is combined with a second thermoplastic polyurethane polymers, such as Elastollan 1385 or a polymer having similar properties to such thermoplastic polyurethane (i.e. Elastollan 1385 type TPU). In some embodiments, the Elastollan 1385 type polymer improves the waterproof properties. The weight ratio of Elastollan 1170 type TPU to Elastollan 1385 type TPU may be at least 1:1, 2:1, 3:1, 4:1 ranging up to 10:1. In some embodiment, the weight ratio of Elastollan 1170 type TPU to Elastollan 1385 type TPU is no greater than 9:1, 8:1, 7:1, 6:1, 5:1, or 4:1.

### Copolymer of Ethylene and Polar Comonomer

In some embodiments, one or more foam layers comprise a copolymer of ethylene and one or more polar comonomers. Suitable polar comonomers include for example vinyl acetate, acrylic acid (meth)acrylate ethyl acrylate, glycidyl methacrylate and anhydride functional monomer. In some embodiments, the copolymer of ethylene and one or more polar comonomers is a terpolymer of at least two polar monomers or a terpolymer of at least one polar monomer and at least one nonpolar monomer other than ethylene. Nonpolar monomers include, for example, propylene, butene, hexene, octene, and combinations thereof. The inclusions of the copolymer ethylene and one or more polar comonomers can improve the waterproof properties of the foam.

In some embodiments, the polar monomer of the ethylene copolymer comprises acrylic acid or methacrylate acid. Ethylene/acrylic acid copolymers are available from various suppliers such as Dow under the trade designation PRIMACOR 1410 or 3460. Ethylene/methacrylic acid copolymers are available from various suppliers such as DuPont Packaging and Industrial Polymers under the trade designations NUCREL 0403 and 0903. Ethylene/vinyl acetate copolymers are available from many suppliers including for example Dow, Dupont and Exxon Chemicals.

In some embodiments, the polar monomer of the ethylene copolymer comprises two or more polar comonomers. Examples of such ethylene-containing copolymers include carbon monoxide modified ethylene/vinyl acetate or anhydride modified ethylene/vinyl acetate. Such ethylene-containing copolymers are commercially available from various suppliers including DuPont Packaging and Industrial Polymers under the trade designations BYNEL E418 and ELVALOY 741.

In some embodiments, the polar monomer of the ethylene copolymer is methyl acrylate.

Physical properties of suitable ethylene methyl acrylate copolymers are described in the following table:

| Acrylate Copolymer | ELVALOY AC1609 | | ELVALOY 1224 | |
|---|---|---|---|---|
| Methyl Acrylate | 9% | by weight | 24% | by weight |
| Density (ASTM D792) | 0.93 | g/cc | 0.944 | g/cc |
| Melt Flow Index | 6 | g/10 min | 2 | g/10/min |
| (190° C./2.16 kg, ASTM | | | | |
| D1238) | | | | |
| Melting Point (ASTM | 101° C. | (213.8° F.) | 91° C. | (195.8 F.) |
| D3418) | | | | |
| Vicat Softening Point | 70° C. | (158° F.), | 48° C. | (118.4° F.) |
| (ASTM D1525) | | | | |

In some embodiments, the ethylene methyl acrylate copolymer comprises (e.g. methyl acrylate) polar comonomer in an amount of at least 5, 10, 15, 20 or 25% by weight, based on the total weight of the ethylene (e.g. methyl acrylate) copolymer. The amount of (e.g. methyl acrylate) comonomer in the foam can be calculated based on the total weight of the components.

In some embodiments, the ethylene (e.g. methyl acrylate) copolymer has melting point of at least 85, 90, 95, or 100° C. In some embodiments, the ethylene (e.g. methyl acrylate) copolymer has melting point of no greater than least 110 or 105° C.

The melt flow is an indication of the molecular weight. In some embodiments, the ethylene (e.g. methyl acrylate) copolymer has a melt flow index of less than 10, 9, 8, 7, 6, 5, 4, 3 g/10 min.

In some embodiments, a blend of at least one thermoplastic polyurethane (TPU) polymer and at least one ethylene (e.g. methyl acrylate (EMA)) copolymer is utilized. The weight ratio of TPU to Ethylene Copolymer (e.g. EMA) may range from 1:10 to 10:1. In some embodiments, the weight ratio of TPU to Ethylene Copolymer (e.g. EMA) is least 1:1, 2:1, 3:1, 4:1, 5:1, 6:1, 7:1, 8:1, or 9:1. In some embodiments, the weight ratio of Ethylene Copolymer (e.g. EMA) to TPU is least 1:1, 2:1, 3:1, 4:1, 5:1, 6:1, 7:1, 8:1, or 9:1.

### Expandable/Expanded Microsphere

The one or more foam layers comprise expanded microspheres. Expandable microspheres typically comprise a thermoplastic barrier shell encapsulating a fluid (e.g., liquid isobutane or isobutene). The thermoplastic shell of the microsphere, having a spherical shape, maintains the encapsulated fluid. When the thermoplastic shell is heated above its glass transition temperature (i.e., the onset temperature of the microsphere), the shell softens and the encapsulated fluid changes from a liquid to a gaseous state, thus dramatically expanding the volume of the microsphere. When expanded, the microspheres have a diameter 3.5 to 4 times their original diameter and a corresponding increase in expanded volume.

The expansion of the expandable microsphere occurs at a temperature range including an onset temperature range of the expandable microspheres. The expandable microspheres typically have a minimum onset temperature of at least 80° C., 90° C., 100° C., 110° C., 120° C., or 130° C. In some embodiments, the minimum onset temperature of the expandable microspheres is no greater than 180° C., 170° C., 160° C., or 150° C. The onset temperature range is typically 5-10 degrees greater than the minimum onset temperature. For example, Expancel 950 MB 80 is reported to have an onset temperature of 138-148° C.

The expansion of the expandable microsphere may also be defined by a temperature range maximum. For example, Expancel 950 MB 80 is reported to have a maximum temperature range of 188-200° C. Thus, the maximum temperature range has a minimum of 188° C. and a maximum of 200° C. In some embodiments, the maximum temperature range of the expandable microspheres has a maximum temperature of no greater than 235° C., 225° C., 215° C., or 205° C. In some embodiments, the maximum temperature range of the expandable microspheres has a minimum temperature of no greater than 215° C., 205° C., 200° C., or 190° C. The (e.g. coextrusion) thermal processing temperature of the thermoplastic (e.g. polyurethane) material is typically greater than the onset range temperature of the expandable microsphere. In typical embodiments, the (e.g. coextrusion) thermal processing temperature of the thermoplastic (e.g. polyurethane) material is no greater than the minimum temperature of the maximum temperature range temperature of the expandable microsphere.

The expandable microspheres can have any suitable average diameter. In some embodiments, the diameter is significantly less than the thickness of a (e.g. coextruded) foam layer. For example, the diameter of the microsphere can be at least 5, 10, or 15 micrometers. In some embodiments, the diameter can be no greater than 50, 45, 40, 35, 30, or 25 microns.

Expandable microspheres are commercially available, for example, from Akzo Nobel under the Expancel family trade name.

The expanded microspheres are typically present in one of more foam layers in an amount of at least 1, 2, 3, 4, 5, 6, 7, 8, 9, or 10 wt. % of a foam layer. In some embodiments, the expanded microspheres are present in an amount no greater than 25, 20, 15, or 10 wt. % of a foam layer. In some embodiments, the expanded microspheres are homogenously distributed within a foam layer.

### Chemical Blowing Agent

In some embodiments, a blowing agent is added to the thermoplastic composition prior to thermal extrusion. The blowing agent may either be an exothermic or endothermic blowing agent, or a combination thereof.

Examples of suitable chemical blowing agents include gaseous compounds such as nitrogen or carbon dioxide, gas (e.g. CO₂) hydrides, carbides such as alkaline earth and alkali metal carbonates and bicarbonates e.g. sodium bicarbonate and sodium carbonate, ammonium carbonate, diaminodiphenylsulphone, hydrazides, malonic acid, citric acid, sodium monocitrate, ureas, azodicarbonic methyl ester, diazabicylooctane and acid/carbonate mixtures. Preferred endothermic blowing agents comprise bicarbonates or citrates.

Examples of suitable physical blowing agents include volatile liquids such as chlorofluorocarbons, partially halogenated hydrocarbons or non-halogenated hydrocarbons like propane, n-butane, isobutane, n-pentane, isopentane and/or neopentane.

Preferred endothermic blowing agents are the so-called “HYDROCEROL” blowing agents as disclosed in a EP-A 158212 and EP-A 211250.

In some embodiments, the foam is free of azodicarbonamide blowing agents.

The total amount of blowing agent is typically at least 0.1, 0.5 or 1 wt. % based on the total weight of the thermoplastic composition. In some embodiments, the total amount of blowing agent is no greater than 5, or 4 wt. % based on the total weight of the thermoplastic composition.

### Method of Making

The polymeric multilayer material described herein can be formed, for example, by a coextrusion and blown film process. The process can use an annular die to a form a molten tube of film oriented radially via air pressure in a “bubble” that is pulled lengthwise in the molten area to bring the film to a final desired thickness. Such method is known and described in previously cited U.S. Ser. No. 11/097,508.

The coextrusion can be run at any suitable temperature. In some embodiments, the extrusion can be run at a temperature ranging from 175° C. to 220° C. (in some embodiments, 180° C. to 210° C., 185° C. to 200° C., or even 190° C. to 210° C.). Further, foam layers or skin layers can be formed by coextruding those layers from additional feedstocks (e.g., a, fourth, fifth, sixth, or seventh feedstock).

The extrusion rate of an individual layer can vary spending on the size of the extruder and the desired thickness of the layer. In some embodiments, the extrusion rate was in the range of 30 to 100 revolutions per minutes.

In typical embodiments, the one or more layer of foam are coextruded between outer (e.g. polyethylene) layers. The outer layers are typically removed.

### Properties/Medical Articles

The foam layer(s) typically have a total thickness of at least 10, 15, or 20 mils. In some embodiments, the foam layer(s) have a total thickness of no greater than 50, 45, 40, 35, 30 or 25 mils.

Due to inclusion of the expanded microspheres and chemical blowing agent, the foam has a plurality of cells that reduce the density, as compared to the density of the thermoplastic polymer(s) from which the foam was prepared from. In some embodiments, the density of the foam is no greater than 0.7, 0.6, or 0.5 g/cc. In some embodiments, the density of the foam is no greater than 0.4, 0.3, or 0.2 g/cc. In some embodiments, the density of the foam is at least 0.1, 0.2, 0.3 g/cc.

In some embodiments, the foam layer(s) have a sufficiently high dry moisture vapor transmission rate (MVTR). In some embodiments, the foam layer(s) have a dry moisture vapor transmission rate of at least 100, 200, 300, 400, 500, 600 g/m² per 24 hours. In some embodiments, the foam has a MVTR of no greater than 600, 500, 400, 300, or 200 g/m² per 24 hours.

In some embodiments, the foam layer(s) are waterproof, i.e. passes the test further described in the examples.

In some embodiments, the hand tear may be characterized as OK or easy in the downweb and crossweb direction. Further, the hand stretch may also be good.

In some embodiments, the foam may be characterized as “soft”. Shore Hardness of the foam is one way to characterize the softness. In some embodiments, the foam has a Shore A hardness ranging from 40-50. The softness and resiliency of the foam can also be characterized according to the indentation force deflection test (IFD), ASTM D3574-08; and constant deflection compression set, ASTM D3574-08.

In some embodiments, the foam has a tensile strength of at least 5, 10, 15, 20 or 25 lbs. force. In some embodiments, the tensile strength is no greater than 25, 20, 15 or 10 lbs. force. In some embodiments, the foam has an elongation of at least 25, 50, 75, or 100%. In other embodiments, the foam has an elongation of at least 200, 300, 400, 500, 600, or 700%. In some embodiments, the elongation is no greater than 700, 600, 500, 400, 300, 200%.

In some embodiments, the foam properties described herein are tested with respect to a piece of foam having a thickness of 20 mils+/−2 mils.

In typical embodiments, the foam is not absorbent and thus does not comprise super absorbent polymer.

### Adhesive

In some embodiments, the article comprises a pressure sensitive adhesive disposed on the foam layer(s). The pressure sensitive adhesive may be (temporarily) covered by a release liner (until use).

Exemplary pressure sensitive adhesives include an acrylic-based pressure sensitive adhesive (i.e. prepared from polymerization of alkyl (meth)acrylate monomers including low Tg monomers), a rubber-based pressure sensitive adhesive, a vinyl alkyl ether pressure sensitive adhesive, a silicone pressure sensitive adhesive, a polyester pressure sensitive adhesive, a polyamide pressure sensitive adhesive, a urethane pressure sensitive adhesive, a fluorinated pressure sensitive adhesive, an epoxy pressure sensitive adhesive, a block copolymer-based pressure sensitive adhesive, or a combination thereof.

Suitable adhesives include acrylate copolymers described in U.S. Pat. No. RE 24,906. Particularly preferred is a 97:3 iso-octyl acrylate:acrylamide copolymer. Also preferred is an 70:15:15 isooctyl acrylate:ethyleneoxide acrylate:acrylic acid terpolymer, as described in U.S. Pat. No. 4,737,410 (Example 31). Other useful adhesives are described in U.S. Pat. Nos. 3,389,827, 4,112,213, 4,310,509, 4,323,557, and 5,849,325.

Other suitable pressure sensitive adhesives include silicone based adhesives. Silicone adhesives are able to effectively secure dressings to skin and upon removal from the skin produce little or no skin damage. An example of a suitable silicone adhesive is disclosed in PCT Publications WO2010/056541 and WO2010/056543.

Various pressure sensitive adhesives that are suitable as medical grade skin-contact adhesive are known. In some embodiments, such medical grade skin-contact adhesive are acrylic or silicone adhesives. Medical grade adhesives that are intended for skin contact are in compliance with FDA Regulation number 880.5240 (e.g. product code KGX).

Suitable pressure sensitive adhesives may be made via a wide variety of techniques. They may include an emulsion pressure sensitive adhesive, a solvent-borne pressure sensitive adhesive, a photo-polymerizable pressure sensitive adhesive, a hot melt pressure sensitive adhesive (i.e., hot melt extruded pressure sensitive adhesive), or a combination thereof.

Coatable hot melt adhesive can then be delivered out of a film die, subsequently contacting the drawn adhesive to a moving plastic web or other suitable substrate. A related coating method involves extruding the coatable hot melt adhesive and a coextruded backing material from a film die and cooling the layered product to form an adhesive tape. Other forming methods involve directly contacting the coatable hot melt adhesive to a rapidly moving plastic web or other suitable preformed substrate. Using this method, the adhesive blend is applied to the moving preformed web using a die having flexible die lips, such as a rotary rod die. After forming by any of these continuous methods, the adhesive films or layers can be solidified by quenching using both direct methods (e.g., chill rolls or water baths) and indirect methods (e.g., air or gas impingement).

### EXAMPLES

TABLE 1
Materials

| Tradename | Description | Supplier |
|---|---|---|
| Dow LDPE 640I | Low Density (0.920 ASTM D792) | Dow |
| | Polyethylene | |
| | Melt Index (190° C./2.16 kg) - | |
| | 2 g/10 min ASTM D1238 | |
| Elastollan ™ 1170A | Thermoplastic Polyether- | BASF |
| (TPU 1170) | Polyurethane | |
| Elastollan ™ 1385A | Thermoplastic polyether and | BASF |
| (TPU 1385) | polyester polyurethane | |
| Elvaloy ™ 1224 | Ethylene methyl acrylate | Dow |
| (EMA 1224) | copolymer | |
| Hydrocerol ™ CF40E | Chemical blowing agent | Avient |
| (Hydro) | | |
| Expancel 950 MB 80 | Expandable microspheres | Nuoryon |
| (EMS) | | |

### Test Methods

MVTR—The “dry” moisture vapor transmission rate (MVTR) was measured according to ASTM E-96-80 using a modified Payne cup method. Specifically, a sample (3.5-cm diameter) was placed between adhesive-containing surfaces of two foil adhesive rings, with an elliptical opening having an area of 0.798 square inches (5.1462×10⁻⁴ square meters). The openings of each ring were carefully aligned. Finger pressure was used to form a foil/sample/foil assembly that was flat, wrinkle-free, and had no void areas in the exposed sample.

A 120-mid glass jar was filled to the halfway level with deionized water. The jar was fitted with a screw-on cap having a 3.8-cm diameter hole in the center thereof and with a 4.45-cm diameter rubber washer having a 2.84-cm diameter hole in its center. The rubber washer was placed on the lip of the jar and the foil/sample assembly was placed on the rubber washer. The lid was then screwed loosely on the jar.

The assembly was placed in a chamber at 38° C. and 20% relative humidity for four hours. At the end of four hours, the cap was tightened inside the chamber so that the sample was level with the cap (no bulging) and the rubber washer was in proper seating position.

The foil/sample assembly was then removed from the chamber and weighed immediately to the nearest 0.01 grams (initial weight W1). The assembly was then returned to the chamber for at least 18-24 hours, after which it was removed and weighed immediately to the nearest 0.01 gram (final weight W2). The MVTR in grams of water vapor transmitted per square meter of sample area in 24 hours was calculated according to the following formula:

$$MVTR ( g / {m}^{2} / 24 hours ) = \frac{( W 1 - W 2 ) * 24}{A * T}$$

wherein A is the exposed sample area (5.1462×10⁻⁴ square meters) and T is the time (hours) in the chamber.

When the sample is in the chamber for 24 hours, the equation is (W1−W2)/A

When the sample is in the chamber for a different time, dividing 24 by the exposed sample area (5.1462×10⁻⁴) results in a constant and the equation is:

$$MVTR ( g / {m}^{2} / 24 hours ) = \frac{( W 1 - W 2 ) * 4.66 \times {10}^{4}}{T}$$

Three measurements of each sample were made, and the average value taken. The MVTR values are reported as of g/m²/24 hrs.

Density—A pycnometer was used to measure the density of each foamed specimen. The buoyancy force was measured according to ASTM D3575-14 (2014) (“Suffix W—test method B), using a pycnometer (obtained under the trade designation “DELTA RANGE” (Model AG204) from Mettler-Toledo, LLC, Columbus, OH). The density was then calculated using Archimedes' principal. That is, samples were cut from the foam film and first weighed dry (m_{dry}). The samples were then placed underwater (de-ionized water) to measure the buoyant force (m_{buoyant}) on the pycnometer. Using the formula below, and knowing the density of water is 1 g/cm³, the density of the foamed samples was calculated.

$${ρ}_{foam} = {ρ}_{water} ( \frac{{m}_{dry}}{{m}_{dry} - {m}_{buoyant}} )$$

Hardness Test—The Shore Durometer Hardness Test (ASTM D-2240 (2000)) was used to measure hardness. Using testing equipment commercially obtained as “MODEL #8 SHORE A” from Pacific Transducer Corp (PTC Instruments), Los Angeles, California.

Tensile Test—Tensile testing was performed using a tensile tester (obtained under the trade designation Instron from Instron, Norwood, MA. For each Example and Comparative Example, test specimens were cut so that testing could be performed in the downweb (or machine) direction and in the crossweb (or transverse) direction. Test specimens were cut to be 1 inch (2.54 cm) in width. The gauge length was 5.0 cm. Depending on the value measured (i.e., downweb or crossweb tensile strength) clamps were attached to downweb ends or crossweb ends of the specimen. Crosshead speed was 20.0 cm/min. For both downweb and crossweb specimens, the maximum force achieved during the test, and the strain at the force maximum, was recorded. For each Example and Comparative Example, in each of the two directions, three specimens were tested.

### Waterproof—Based on EN 13726-3 Hydrostatic Head Test

Waterproofness was performed using a hydrostatic head test (obtained under the trade designation TEXTESTER from TEXTEST Instruments, Schwerzenbach, Switzerland). For each Example and Comparative Example, test specimens were cut to be 6″ (15.25 cm) by 6″ (15.25 cm). Close drain, place test specimen on the lower ring, cover the upper surface of the specimen with dry filter paper larger than the test area, clamp the specimen in the test head and fill the instrument with deionized water. Set to static test and 49 mbar pressure for 5 minutes. Observe the specimen, if 5 leaks appear, the test can be stopped early, otherwise allow the test to run for 5 minutes. Inspect the filter paper for any wetness/leaks. Any wetness on the filter paper results in a failure for waterproofness.

Hand Tear—For each Example and Comparative Example, test specimens were cut so that testing could be performed in the downweb (or machine) direction and in the crossweb (or transverse) direction. Test specimens were cut to be 1 inch (2.54 cm) in width and 4 inches (10.16 cm) in length. Pinch the specimen with each hand's forefinger and thumb in the middle of the sample, at about 2 inches (5.08 cm), and proceed to tear the specimen. The specimens were rated by very tough (significant effort to tear or could not tear), ok (low to medium effort to tear), and easy (very low effort to tear).

Stretch—For each Example and Comparative Example, test specimens were cut so that testing could be performed in the downweb (or machine) direction. Test specimens were cut to be 1 inch (2.54 cm) in width and 4 inches (10.16 cm) in length. Pinch the specimen with forefinger and thumb at each end of the specimen and proceed to stretch the specimen about 10-12 inches/minute. The specimens were rated stretchy (high elastic stretch), good (some elastic stretch), and little (specimen snapped when stretched).

### Preparation of Samples

A seven layer coextruded material was produced with a seven layer pancake stack die (Type LF-400 Coex 7-layer from Labtech Engineering Co. Ltd., Thailand) as described in U.S. Pat. No. 11,097,508. The outer layers comprised Dow LDPE 640i. The five interior layers comprised the composition indicated in forthcoming Table 2.

Airflow to the die was manually controlled to achieve a blow up ratio of approximately 2:1. The bubble was subsequently collapsed approximately ten feet above die and rolled up. The expandable microspheres and chemical blowing agent were fed by 7 independent 20 mm diameter extruders with an approximately 30:1 L/D and a compression ratio of 2:1. The following process conditions were utilized:
Layers 1 & 7 Extruder Temperatures: Zones 1, 2 and 3: 360° F. (182° C.)
Layers 2-6 Extruder Temperatures: Zones 1, 2 and 3: 360° F.
The Adaptor and Die Temperatures: Adaptor 340-360° F.

The composition of the layers, thickness, and test results as further described in the following Tables 2-5:

TABLE 2

| | Ex. 1 | Ex. 2 | Ex. 3 | Ex. 4 |
|---|---|---|---|---|
| Layer 1 | 100% LDPE 640i | | | |
| Thickness | 2 mil | 2 mil | 3 mil | 3 mil |
| Layer 2 | TPU 1170 (93)/ | TPU 1170 (93)/ | TPU 1170 (93)/ | TPU 1170 (93) |
| | Hydro (4)/ | Hydro (4)/ | Hydro(4)/ | (80)/TPU 1385 |
| | EMS (3) | EMS(3) | EMS (3) | (20)/Hydro (4)/ |
| | | | | EMS(3) |
| Layer 3 | TPU 1170 (75)/ | TPU 1170 (50)/ | TPU 1170 (25)/ | TPU 1170 (50)/ |
| | EMA 1224 (25)/ | EMA 1224 (50)/ | EMA 1224 (75)/ | EMA 1224 (50)/ |
| | Hydro (5)/ | Hydro (5)/ | Hydro (5)/ | Hydro (5)/ |
| | EMS (10) | EMS (10) | EMS (10) | EMS (10) |
| Layer 4 | TPU 1170 (75)/ | TPU 1170 (50)/ | TPU (25)/ | TPU 1170 (50)/ |
| | EMA 1224 (25)/ | EMA 1224 (50)/ | EMA 1224 (75)/ | EMA 1224 (50)/ |
| | Hydro (5/ | Hydro (5)/ | Hydro (5)/ | Hydro (5)/ |
| | EMS (10) | EMS (10) | EMS (10) | EMS (10) |
| Layer 5 | TPU 1170 (75)/ | TPU 1170 (50)/ | TPU 1170 (25)/ | TPU 1170 (50)/ |
| | EMA 1224 (25)/ | EMA 1224 (50)/ | EMA 1224 (75)/ | EMA 1224 (50)/ |
| | Hydro (5)/ | Hydro (5)/ | Hydro (5)/ | Hydro (5)/ |
| | EMS (10) | EMS (10) | EMS (10) | EMS (10) |
| Layer 6 | TPU 1170/ | TPU 1170/ | TPU 1170/ | TPU 1170 (80)/ |
| | Hydro(4)/ | Hydro (4)/ | Hydro (4)/ | 1385 (20)/ |
| | EMS(3) | EMS (3) | EMS (3) | Hydrocerol (4)/ |
| | | | | EMS (3) |
| Layer 7 | 100% LDPE 640i | | | |
| Thickness | 2 mil | 2 mil | 2 mil | 2 mil |

TABLE 3

| | Ex. 1 | Ex. 2 | Ex. 3 | Ex. 4 |
|---|---|---|---|---|
| Total Thickness of Foam | 22.58 | 20.5 | 21.75 | 21.92 |
| (i.e. sum of Layers 2-6) | | | | |
| (mils) | | | | |
| Grams/square meter (GSM) | 171 | 170 | 156 | 155 |
| of foam (without | | | | |
| layers 1 and 7) | | | | |
| MVTR (g/m²/day) | 157 | 146 | 131 | 144 |
| Waterproof | PASS | PASS | PASS | PASS |
| Durometer Shore A Hardness | 45 | 45 | 43 | 45 |
| CW Hand Tear | Very Tough | OK | Easy | Easy |
| DW Hand Tear | Very Tough | Easy | Easy | Easy |
| Hand Stretch | Stretchy | Good | Little | Little |
| Foam Density (g/cm³) | 0.323 | 0.337 | 0.291 | 0.309 |
| Tensile (lbf) DW | 10.90 | 9.15 | 5.25 | 5.35 |
| Tensile (lbf) CW | 6.90 | 4.87 | 4.07 | 4.26 |
| Elongation (%) DW | 642 | 632 | 25 | 27 |
| Elongation (%) CW | 505 | 351 | 24 | 26 |

TABLE 4

| | Ex. 5 | Ex. 6 | Ex. 7 | Ex. 8 |
|---|---|---|---|---|
| Layer 1 | 100% LDPE 640i | | | |
| Thickness | 3 mil | 3 mil | 3 mil | 3 mil |
| Layer 2 | TPU 1170/ | TPU 1170/ | TPU 1170/ | Elastollan 1170 (80)/ |
| | Hydro(4)/ | Hydro(4)/ | Hydro (4)/ | Elastollan 1385(20)/ |
| | EMS(3) | EMS(3) | EMS(3) | Hydrocerol(4)/Akzo (3) |
| Layer 3 | TPU 1170 (60)/ | TPU 1170(65)/ | TPU 1170 (25)/ | Elastollan 1170 (80)/ |
| | EMA 1224 (40)/ | EMA 1224 (35)/ | EMA 1224 (75)/ | Elastollan 1385(20)/ |
| | Hydro (5)/ | Hydro (5)/ | Hydro (5)/ | Hydrocerol(4)/Akzo (3) |
| | EMS (10) | EMS (10) | EMS (10) | |
| Layer 4 | TPU 1170 (60)/ | TPU 1170 (65)/ | TPU 1170 (25)/ | Elastollan 1170 (80)/ |
| | EMA 1224 (40)/ | EMA 1224 (35)/ | EMA 1224 (75)/ | Elastollan 1385(20)/ |
| | Hydro (5)/ | Hydro (5)/ | Hydro (5)/ | Hydrocerol(4)/Akzo (3) |
| | EMS (10) | EMS (10) | EMS (10) | |
| Layer 5 | TPU 1170 (60)/ | TPU 1170 (65)/ | TPU 1170 (25)/ | Elastollan 1170 (80)/ |
| | EMA 1224 (40)/ | EMA 1224 (35)/ | EMA 1224 (75)/ | Elastollan 1385(20)/ |
| | Hydro (5)/ | Hydro (5)/ | Hydro (5)/ | Hydrocerol(4)/Akzo (3) |
| | EMS (10) | EMS (10) | EMS (10) | |
| Layer 6 | TPU 1170/ | TPU 1170/ | TPU 1170/ | Elastollan 1170 (80)/ |
| | Hydrocerol (4)/ | Hydrocerol (4)/ | Hydrocerol (4)/ | Elastollan 1385(20)/ |
| | EMS (3) | EMS (3) | EMS (3) | Hydrocerol(4)/Akzo (3) |
| Layer 7 | TPU 1170 | TPU1170 | TPU 1170 | 640i |

TABLE 5

| | | | | | Comparative |
|---|---|---|---|---|---|
| | | | | | Polyvinyl |
| | | | | | chloride |
| | Ex. 5 | Ex. 6 | Ex. 7 | Ex. 8 | Foam |
| Total Thickness of | 20.25 | 19.67 | 20.5 | 18 | 22 |
| Foam (mils) | | | | | |
| Grams/square meter | 184 | 172 | 171 | 211 | 182 |
| (GSM) of foam | | | | | |
| (without layers | | | | | |
| 1 and 7) | | | | | |
| MVTR (g/m²/day) | 144 | 146 | 131 | 469 | 627 |
| Waterproof | PASS | PASS | PASS | PASS | PASS |
| Durometer Shore A | 46 | 46 | 45 | 50 | 31 |
| Hardness | | | | | |
| CW Hand Tear | Very | OK | OK | Very | Easy |
| | Tough | | | Tough | |
| DW Hand Tear | Very | Easy | OK | Very | Easy |
| | Tough | | | Tough | |
| Hand Stretch | Stretchy | Good | Good | Stretchy | Good |
| Foam Density | 0.366 | 0.393 | 0.362 | 0.525 | 0.324 |
| (g/cm³) | | | | | |
| Tensile (lbf) DW | 13.5 | 8.7 | 7.5 | 18.3 | 4.1 |
| Tensile (lbf) CW | 7.24 | 8.66 | 4.10 | 15.7 | 4.4 |
| Elongation (%) DW | 671 | 616 | 531 | 591 | 213 |
| Elongation (%) CW | 581 | 670 | 26 | 569 | 205 |

The results show that the exemplified foam can have comparable and improved properties in comparison to PVC foam.

What is claimed is:

1. An article comprising:
a foam substrate comprising
a thermoplastic composition comprising polyurethane, a copolymer of ethylene and a polar comonomer, or a combination thereof; and
expanded microspheres; and
wherein the article is a medical tape, bandage, dressing, or wrap.

2. The article of claim 1 wherein the foam has a density ranging from 0.2 to 0.6 g/cc.

3. The article of claim 1 wherein the foam has a MVTR of at least 100, 200, 300, 400, 500, or 600 g/m² per 24 hours.

4. The article of claim 1 wherein the foam has a Shore A hardness ranging from 40-60.

5. The article of claim 1 wherein the foam is waterproof.

6. The article of claim 1 wherein the thermoplastic composition comprises polyurethane and a copolymer of ethylene and a polar comonomer.

7. The article of claim 1 wherein the copolymer of ethylene and a polar comonomer comprises methyl acrylate in an amount ranging from 15 to 30 wt. % based on the total weight of the copolymer.

8. The article of claim 1 wherein the expanded microspheres are present in an amount ranging from 1 to 20 wt. % of the foam.

9. The article of claim 1 wherein the foam has a thickness ranging from 10 to 50 mils (250 microns to 1250 microns).

10. The article of claim 1 wherein the foam is free of halogens and azodicarbonamide blowing agents.

11. The article of claim 1 further comprising a pressure sensitive adhesive skin contact adhesive disposed on a major surface of the foam substrate.

12. The article of claim 11 wherein the adhesive is an acrylic adhesive or silicone adhesive.

13. A method of use comprising:
providing an article according to claim 1; and
contacting the foam and/or pressure sensitive adhesive with skin.

14. A method of making a foam article comprising
providing a composition comprising
a thermoplastic polymer comprising polyurethane, a copolymer of ethylene and a polar comonomer, or a combination thereof; and
expandable microspheres having a minimum onset temperature;
extruding the composition at a temperature greater than the minimum onset temperature of the expandable microspheres.

15. The method of claim 14 wherein the composition further comprises a blowing agent.

16. The method of claim 14 wherein one or more layers of the composition are coextruded between polyolefin outer layer layers.

17. The method of claim 16 further comprising removing at least one outer layer and applying a layer of pressures sensitive adhesive to foam.

18. An article comprising:
a foam substrate comprising:
a thermoplastic polymer comprising polyurethane, a copolymer of ethylene and a polar comonomer, or a combination thereof; and
expanded microspheres; and
a pressure sensitive adhesive layer disposed on a outermost major surface of the foam substrate.

19. An article comprising:
foam, wherein the foam comprises a thermoplastic polymer comprising polyurethane, a copolymer of ethylene and a polar comonomer, or a combination thereof; expanded microspheres; and chemical blowing agent.
